(19) United States
(12) Patent Application Publication
(10) Pub. No.: US 2025/0265679 A1
(43) Pub. Date: Aug. 21, 2025

(54) SPECTRAL COMPUTED TOMOGRAPHY WITH DISENTANGLED REPRESENTATION

(71) Applicant: GE Precision Healthcare LLC, Waukesha, WI (US)

(72) Inventors: Pengwei Wu, Clifton Park, NY (US); Bruno De Man, Clifton Park, NY (US)

(73) Assignee: GE Precision Healthcare LLC, Waukesha, WI (US)

(21) Appl. No.: 18/581,720

(22) Filed: Feb. 20, 2024

(51) Int. Cl. G06T 5/50; G06T 11/00

(52) U.S. Cl. CPC G06T 5/50; G06T 11/005; G06T 11/008; G06T 2207/10081; G06T 2207/20081; G06T 2207/20084; G06T 2207/30004; G06T 2211/441

(57) ABSTRACT

A system for generating target images, comprising a processor configured to train a latent space encoder to generate latent space information by encoding the multi-spectral images to generate anatomy latent space information and contrast latent space information, the anatomy latent space information representing compressed anatomy information in the multi-spectral images, the contrast latent space information representing compressed contrast information in the multi-spectral images, combining selected features of the anatomy latent space information and the contrast latent space information to reproduce the multi-spectral images, comparing the reproduced multi-spectral images to the multi-spectral images, and adjusting the encoding of the multi-spectral images and repeating the training until. After training the latent space encoder, train an output decoder to generate target images by inputting the anatomy latent space information to a prediction model, predicting target images by the prediction model based on the anatomy latent space information.

### FIELD

A system and method for spectral computed tomography with disentangled representation.

### BACKGROUND

Photon-counting CT (PCCT) is an emerging tomographic imaging technique that offers improved diagnostic performance through better spatial and energy resolution. It uses multiple energy bins to measure to spectral dependence of the X-ray attenuation, similar to dual-energy computed tomography (CT) or any other forms of spectral CT. However, high-quality image reconstruction and material decomposition is a challenging issue due to various sources of image noise and artifacts (e.g., quantum noise, charge sharing, pulse pileup, Compton scatter, beam hardening effects and other issues).

### SUMMARY

In embodiments, the present disclosure relates to a system for generating target images, comprising a database of multi-spectral images, and a processor configured to train a latent space encoder to generate latent space information by encoding the multi-spectral images to generate anatomy latent space information and contrast latent space information, the anatomy latent space information representing compressed anatomy information in the multi-spectral images, the contrast latent space information representing compressed contrast information in the multi-spectral images, combining selected features of the anatomy latent space information and the contrast latent space information to reproduce the multi-spectral images, comparing the reproduced multi-spectral images to the multi-spectral images, and adjusting the encoding of the multi-spectral images and repeating the training until the reproduced multi-spectral images match the multi-spectral images. After training the latent space encoder, train an output decoder to generate target images by inputting the anatomy latent space information to a prediction model, predicting target images by the prediction model based on the anatomy latent space information, comparing the predicted target images to reference target images related to the multi-spectral images, and adjusting weights of the prediction model based on the comparison and repeating the training until the images predicted by the prediction model match the reference target images related to the multi-spectral images.

In embodiments, the processor is configured to generate the latent space information by applying a convolutional neural network to the multi-spectral images to extract and separate anatomy and contrast information from the multi-spectral images.

In embodiments, the processor is configured to select anatomy features as common anatomy features between the anatomy latent space information.

In embodiments, the processor is configured to combine the anatomy latent space information and the contrast latent space information by concatenating the anatomy latent space information and the contrast latent space information.

In embodiments, the processor is configured to compare the reproduced multi-spectral images to the multi-spectral images by computing a loss function of the reproduced multi-spectral images as compared to the multi-spectral images and repeating the training until the loss function is less than a loss function threshold.

In embodiments, the multi-spectral images are images of anatomy captured by medical imaging devices from a common frame of reference relative to the anatomy and operating at different spectral frequencies.

In embodiments, the processor is configured to average the anatomy latent space information prior to inputting the anatomy latent space information to the prediction model.

In embodiments, the prediction model is a neural network that predicts the target images and in supervised manner compares the predicted target images to reference target images related to the multi-spectral images to determine corrective measures to adjust the weights of the prediction model which are neural network weights.

In embodiments, the target images are medical images related to states of human anatomy and comprise one or more of virtual monoenergetic images, virtual non-contrast images, material maps and downstream segmentations.

In embodiments, the processor is configured to utilize the trained latent space encoder to generate new latent space information for new multi-spectral images and input the new latent space information to the trained output decoder to generate new target images.

A method for generating target images, comprising training, by a processor, a latent space encoder to generate latent space information by encoding multi-spectral images to generate anatomy latent space information and contrast latent space information, the anatomy latent space information representing compressed anatomy information in the multi-spectral images, the contrast latent space information representing compressed contrast information in the multi-spectral images, combining selected features of the anatomy latent space information and the contrast latent space information to reproduce the multi-spectral images, comparing the reproduced multi-spectral images to the multi-spectral images, and adjusting the encoding of the multi-spectral images and repeating the training until the reproduced multi-spectral images match the multi-spectral images. After training the latent space encoder, training, by the processor, an output decoder to generate target images by inputting the anatomy latent space information to a prediction model, predicting target images by the prediction model based on the anatomy latent space information, comparing the predicted target images to reference target images related to the multi-spectral images, and adjusting weights of the prediction model based on the comparison and repeating the training until the images predicted by the prediction model match the reference target images related to the multi-spectral images.

In embodiments, the method comprises generating, by the processor, the latent space information by performing convolutions on the multi-spectral images to extract and separate anatomy and contrast information from the multi-spectral images.

In embodiments, the method comprises selecting, by the processor, anatomy features as common anatomy features between the anatomy latent space information.

In embodiments, the method comprises combining, by the processor, the anatomy latent space information and the contrast latent space information by concatenating the anatomy latent space information and the contrast latent space information.

In embodiments, the method comprises comparing, by the processor, the reproduced multi-spectral images to the multi-spectral images by computing a loss function of the reproduced multi-spectral images as compared to the multi-spectral images and repeating the training until the loss function is less than a loss function threshold.

In embodiments, the multi-spectral images are images of anatomy captured by medical imaging devices from a common frame of reference relative to the anatomy and operating at different spectral frequencies.

In embodiments, the method comprises averaging, by the processor, the anatomy latent space information prior to inputting the anatomy latent space information to the prediction model.

In embodiments, the prediction model is a neural network that predicts the target images and in supervised manner compares the predicted target images to reference target images related to the multi-spectral images to determine corrective measures to adjust the weights of the prediction model which are neural network weights.

In embodiments, the target images are medical images related to states of human anatomy and comprise one or more of virtual monoenergetic images, virtual non-contrast images, material maps and downstream segmentations.

In embodiments, the method comprises utilizing, by the processor, the trained latent space encoder to generate new latent space information for new multi-spectral images and input the new latent space information to the trained output decoder to generate new target images.

### BRIEF DESCRIPTION OF THE DRAWINGS

So that the way the above-recited features of the present disclosure may be understood in detail, a more particular description of the disclosure, briefly summarized above, may be made by reference to example embodiments, some of which are illustrated in the appended drawings. It is to be noted, however, that the appended drawings illustrate only example embodiments of this disclosure and are therefore not to be considered limiting of its scope, for the disclosure may admit to other equally effective example embodiments.

FIG. 1 shows a block diagram of training models for spectral computed tomography with disentangled representation, according to an example embodiment of the present disclosure.

FIG. 2 shows a flowchart of training a disentanglement model, according to an example embodiment of the present disclosure.

FIG. 3 shows a flowchart of training a prediction model, according to an example embodiment of the present disclosure.

FIG. 4 shows a flowchart of executing a trained models for spectral computed tomography with disentangled representation, according to an example embodiment of the present disclosure.

FIG. 5 shows example images for spectral computed tomography with disentangled representation, according to an example embodiment of the present disclosure.

FIG. 6 shows a block diagram of hardware devices for the training and execution of spectral computed tomography with disentangled representation, according to an example embodiment of the present disclosure.

FIG. 7 shows a block diagram of components of the training and execution of hardware devices for spectral computed tomography with disentangled representation, according to an example embodiment of the present disclosure.

### DETAILED DESCRIPTION

Various example embodiments of the present disclosure will now be described in detail with reference to the drawings. It should be noted that the relative arrangement of the components and steps, the numerical expressions, and the numerical values set forth in these example embodiments do not limit the scope of the present disclosure unless it is specifically stated otherwise. The following description of at least one example embodiment is merely illustrative in nature and is in no way intended to limit the disclosure, its application, or its uses. Techniques, methods, and apparatus as known by one of ordinary skill in the relevant art may not be discussed in detail but are intended to be part of the specification where appropriate. In all the examples illustrated and discussed herein, any specific values should be interpreted to be illustrative and non-limiting. Thus, other example embodiments may have different values. Notice that similar reference numerals and letters refer to similar items in the following figures, and thus once an item is defined in one figure, it is possible that it need not be further discussed for the following figures. Below, the example embodiments will be described with reference to the accompanying figures.

Spectral computed tomography offers improved diagnostic performance through improved spatial and energy resolution. This disclosure demonstrates a deep learning (DL) based technique to produce representative spectral medical target (e.g., computed tomography (CT)) output images such as virtual monoenergetic images, material maps, and downstream segmentations from multi-spectral input images via a more simplified disentangled representation, which disentangles CT images into patient anatomy (same for all energy levels) and contrast latent spaces (different for all energy levels) in a simplistic unsupervised manner. The patient anatomy space is then decoded into the desired output images through supervised image synthesis. This method is generally described in the CT-image-domain (post reconstruction) but may also be applied in the projection-domain (pre reconstruction). In CT images, the raw measurements (also known as projection data) may be represented in sinograms, which are reconstructed to obtain patient images. Hence, pre-reconstruction methods are methods applied to the sinograms or projection data, while post-reconstruction methods are methods applied to the reconstructed images.

For example, sinograms may be two-dimensional or three-dimensional datasets representing the raw measurements captured by the CT scanner as it rotates around the patient, detecting X-rays that have passed through the body at various angles. Pre-reconstruction techniques involve manipulating these raw data sets to correct for physical phenomena such as beam hardening, scatter, or noise, which can affect the quality of the final image. Algorithms in this phase may also perform tasks such as filtering, normalization, and calibration to prepare the data for the subsequent reconstruction process. The goal of pre-reconstruction processing is to condition the data in a way that enhances the accuracy and quality of the images that will be generated in the next phase.

Post-reconstruction, on the other hand, deals with the data after it has been converted into cross-sectional images through a reconstruction algorithm. These algorithms take the pre-processed sinograms and apply techniques to construct a three-dimensional volume or series of two-dimensional slices that represent the scanned anatomy. Post-reconstruction processing may include various image enhancement techniques, such as denoising, contrast enhancement, and edge sharpening, to improve the diagnostic utility of the images. Additionally, it can involve advanced data driven deep learning methods like the disentangled representation technique described in this disclosure, which separates anatomical and contrast information to facilitate the generation of specialized images for improved medical assessment. Both pre- and post-reconstruction phases are integral to the CT imaging process, each serving distinct roles in the transformation of raw data into clinically useful images. The disclosed disentanglement method is applicable in either of these phases.

Specifically, in the pre-reconstruction domain, the disclosed technique would be applied to the raw projection data collected by the CT scanner, which is typically represented in the form of sinograms. These sinograms contain the raw measurements of X-ray attenuation at different energy levels and at various angles around the patient. The application of the technique in this domain may involve the following steps: 1. Disentanglement of Raw Data: The unsupervised learning component of the technique would be used to separate the raw projection data into anatomy and contrast latent spaces. This step would aim to identify and encode the consistent anatomical structures present in the data, while also capturing the varying contrast information that depends on the energy spectrum of the X-rays. 2. Noise and Artifact Reduction: By working with the raw data, the technique could address and correct for various artifacts and noise sources, such as beam hardening, scatter, or electronic noise, which can degrade image quality if not properly managed before reconstruction. 3. Synthesis of Enhanced Sinograms: The disentangled anatomy latent space could then be used to generate enhanced sinograms that are optimized for the subsequent reconstruction process, potentially leading to clearer and more accurate images.

In the post-reconstruction domain, the technique would be applied to images that have already been reconstructed from the sinogram data. The application in this domain may involve: 1. Disentanglement of Reconstructed Images: The technique would analyze the reconstructed images, separating them into anatomy and contrast latent spaces. The anatomy latent space would represent the structural information of the tissues, while the contrast latent space would reflect the differences in X-ray attenuation at various energy levels. 2. Image Enhancement: The disentangled anatomy latent space would be used to synthesize enhanced images, such as virtual monoenergetic images or material maps, which could provide additional diagnostic information beyond what is available in the standard reconstructed images. 3. Downstream Analysis: The technique could facilitate downstream tasks such as segmentation or identification of specific tissues, by providing a clearer representation of the anatomical structures without the confounding effects of varying contrast levels.

Benefits of the disclosed methods, devices and systems described herein include but are not limited to producing high-quality spectral CT images (including monochromatic images, tissue/material maps, color maps, and segmentations) in a computationally efficient manner. Spectral CT is of clinical interests in numerous applications such as non-invasive diagnosis in obstructive coronary artery disease, coronary atherosclerosis characterization, and non-invasive diagnosis of urolithiasis to name a few. This disclosure is also useful for downstream image analysis task (e.g., segmentation of thin blood vessels). It is noted that although the disclosed systems/methods are described with respect to processing of CT images, it is noted that the systems/methods are also applicable to processing of other types of medical images such as magnetic resonance imaging (MRI), ultrasound, X-rays, and mammography to name a few.

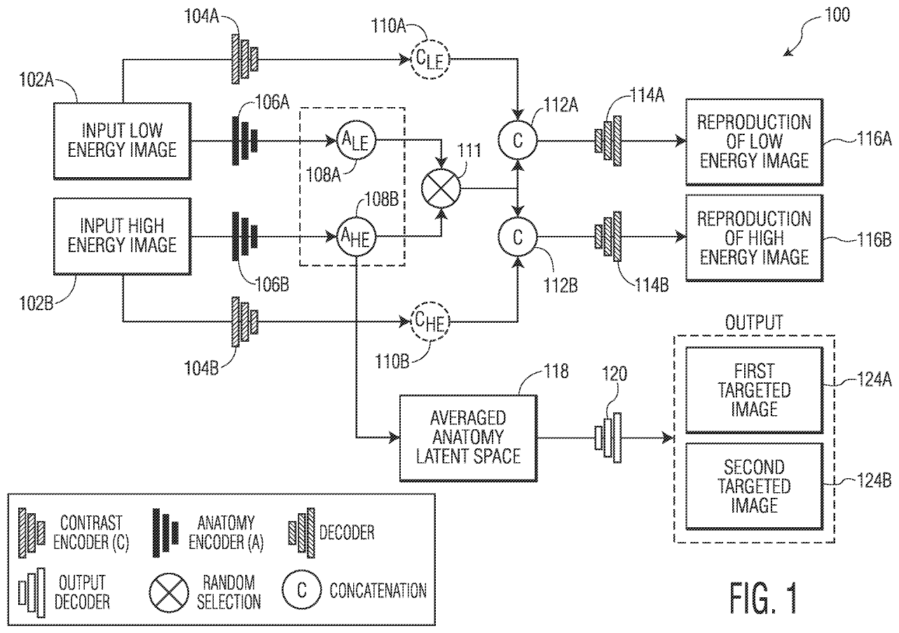

FIG. 1 shows a block diagram 100 of training models for spectral computed tomography with disentangled representation. This technology is a semi-supervised DL approach, which may be divided into two parts: (1) input CT images at different energy levels are first disentangled into “anatomy” and “contrast” latent spaces respectively which may be combined to accurately reproduce the input CT images; and (2) anatomy latent space information are decoded into the targeted output image(s).

Overall, the proposed method takes multiple spectral CT images as input images, encodes them into an anatomy latent representation and a contrast latent representation per input image. The anatomy latent representation is then converted into high-fidelity desired output images. The input images may be images corresponding to different X-ray spectra, different detector bins, any type of energy bins, basis material decomposition images, preliminary monochromatic images, linear combinations of the above, or a set of images including several of the above categories and basis material images. The output images may be monochromatic attenuation images, material density images, material volume fraction images, density images, other physics representation images (e.g., electron density, effective Z, stopping power, . . . ), anatomy segmentation images and basis material images.

This solution can be broken up into two parts as mentioned above. The first part is unsupervised (i.e., there is no label for the latent space). An important aspect here is that anatomy (e.g., the shapes and boundaries) are the common information between input CT images at different energy levels. In spectral imaging, common information symbolizes an artifact-free representation of the underlying tissue materials. In the second part, the disentangled patient anatomy latent space may be decoded into the targeted medical image outcome(s) such as virtual monoenergetic images (VMI), virtual non-contrast images (VNC), material maps, and downstream segmentations. This second part is a supervised image synthesis task. Alternative implementations in the projection domain may also be envisioned, in which case both inputs and outputs are in the projection domain.

Prior to accurately generating the target images, the models (i.e., encoders) can be trained in a semi-supervised manner. Training generally can occur in two phases. In a first phase, a disentanglement model (i.e., encoder) can be trained in an unsupervised manner. For example, multispectral images such as low energy image 102A and high energy image 102B are input into contrast encoders 104A/104B and anatomy encoders 106A/106B. Encoders 106A/106B encode the multi-spectral images to generate anatomy latent space information 108A/108B, while encoders 104A/104B encode the multi-spectral images to generate contrast latent space information 110A/110B. The anatomy latent space information 108A/108B represent compressed anatomy information in the multi-spectral images, while the contrast latent space information 110A/110B represent compressed contrast information in the multi-spectral images (e.g., smaller image, matrix, vector, etc.). For example, the anatomy latent space information may represent maps corresponding to specific human tissues (anatomy information). The encoder (through training) chooses how it will encode the anatomy information. Similarly, the contrast latent space may provide lookup tables to translate (i.e., map) the anatomy back to the target images.

Essentially, the anatomy latent space information represents a distilled, abstracted representation of the patient's anatomical structures as captured by the CT system. This information is generated by an encoder that processes the multi-spectral CT images to isolate and encode the underlying anatomical information that is consistent across different energy levels. The anatomy latent space captures the geometric and spatial information of tissues, bones, and organs, effectively stripping away the variations in X-ray attenuation that are due to differing material properties and the energy spectrum of the X-rays used during the scan. This results in information that emphasize the structural integrity and layout of the patient's anatomy, which is a common factor in images taken at various energy levels. The anatomy latent space serves as a stable foundation for subsequent image synthesis tasks, such as creating virtual monoenergetic images or performing tissue segmentation.

On the other hand, contrast latent space information encode the variable aspects of the CT images that arise from the different energy-dependent X-ray attenuation properties of various materials in the scanned volume. This information is also generated by an encoder but focus on capturing the contrast information that changes with the energy spectrum of the X-rays. This includes the differentiation of materials based on their spectral signatures, such as the identification of iodine contrast agents, calcium deposits, or other artifacts that have distinct attenuation profiles at different energy levels. The contrast latent space provides a complementary set of data that, when combined with the anatomy latent space, can be used to reconstruct the original multi-spectral images.

The solution then can combine selected features of the anatomy latent space information and the contrast latent space information to reproduce the original multi-spectral images. An example combination may select, at block 111, common anatomy features between the anatomy latent space information 108A/108B and the contrast latent space information 110A/110B and then separately concatenate, at blocks 112A/112B, the common features with the contrast latent space information 110A/110B. The combined images are then decoded by decoders 114A/114B to produce reproduced images 116A/116B which are compared to the original multi-spectral images 102A/102B. The system adjusts parameters of encoders 104A/104B and 106A/106B and repeats the training until the reproduced multi-spectral images 116A/116B match the multi-spectral images 102A/102B to a degree (e.g., have an acceptable difference less than a threshold). For example, the difference may be determined by a loss function that measures the information loss due to the image reproduction and compares the information loss to a loss function threshold. The type of loss function used in the training of a spectral CT system with disentangled representation can vary depending on the specific objectives of the model and the characteristics of the data. However, some examples may include but are not limited to Mean Squared Error (MSE) loss which measures the average squared difference between the estimated values and the actual value. In the context of this system, it could quantify the pixel-wise differences between the reproduced multi-spectral images and the original multi-spectral images. Another example may be Mean Absolute Error (MAE) loss which measures the average absolute difference between predicted and actual values. It is less sensitive to outliers than MSE, which might be beneficial if the training data contains anomalies. This process results in accurately training encoders 106A/106B which act as a disentanglement model. In order to facilitate this unsupervised training, each iteration may use a different (i.e., new) set of input multispectral images to update the encoder models until the encoder models accurately reproduce the original input images from the latent space information.

In a second phase, a target image prediction model (i.e., decoder) can be trained in a supervised manner to predict target (i.e., desired) images. For example, after training the latent space encoder, as described above, the target image prediction model is trained to generate the target images by combining the two or more anatomy latent space information 108A/108B into average anatomy latent space information 118 (e.g., averaging, median filtering, or through other ways of combining the anatomy latent space information), passing these images to a decoder 120 that predicts target images 124A/124B, and comparing the predicted target images 124A/124B to known reference target images (not shown) related to the multi-spectral images 102A/102B. As mentioned above, these target images may include VMI images, VNC images, material maps, downstream segmentations and the like. The method adjusts decoder weights of the prediction model based on the above comparison. This process can be repeated for various labeled pairs of anatomy latent space information 108A/108B and reference target images related to the source multi-spectral images 102A/102B until target images 124A/124B predicted by decoder 120 match the reference target images related to the multi-spectral images.

The result of training the disentanglement model in the first phase and training the prediction model in the second phase is that the models may be deployed as a single comprehensive model that receives new multi-spectral images, accurately converts the multi-spectral images to anatomy latent space information 108A/108B (compressed images) which are then be used to accurately predict target images 124A/124B. In other words, the comprehensive model may perform prediction based on simplified anatomy latent space information 108A/108B (compressed images) rather than the more complex source multi-spectral images 102A/102B. This not only produces more accurate predictions, but also reduces resource consumption and computation time when making the predictions, because the amount of data present in the anatomy latent space information 108A/108B is significantly reduced as compared to the date in the multi-spectral images 102A/102B, while maintaining enough information for making accurate predictions.

The disentanglement model, prediction model and/or comprehensive model may be neural networks such as a convolutional neural network (CNN). For example, the disentanglement model may be a neural network in which the multi-spectral images are input to the CNN, disentangled into the anatomy and contrast latent space information which are then combined to reproduce the multi-spectral images. During training, the weights of the CNN are then adjusted by comparing the original multi-spectral images to the reproduced multi-spectral images. Likewise, the prediction model may be a neural network in which the anatomy latent space information are input to the CNN, used to predict the target images. During training, the weights of the CNN are then adjusted by comparing known target images to the generated target images. The comprehensive model may be a comprehensive neural network that combines both the disentanglement CNN and the prediction CNN into a comprehensive CNN in which the multi-spectral images are input to the comprehensive CNN, disentangled into the anatomy latent space information which are used to predict the target images.

It is noted that training may be repeated as needed depending on the accuracy of the trained model. In other words, the trained model may be monitored for model drift over time. If the model drifts beyond an acceptable prediction accuracy, then the model may be retrained on new multi-spectral images to increase accuracy before being updated and deployed.

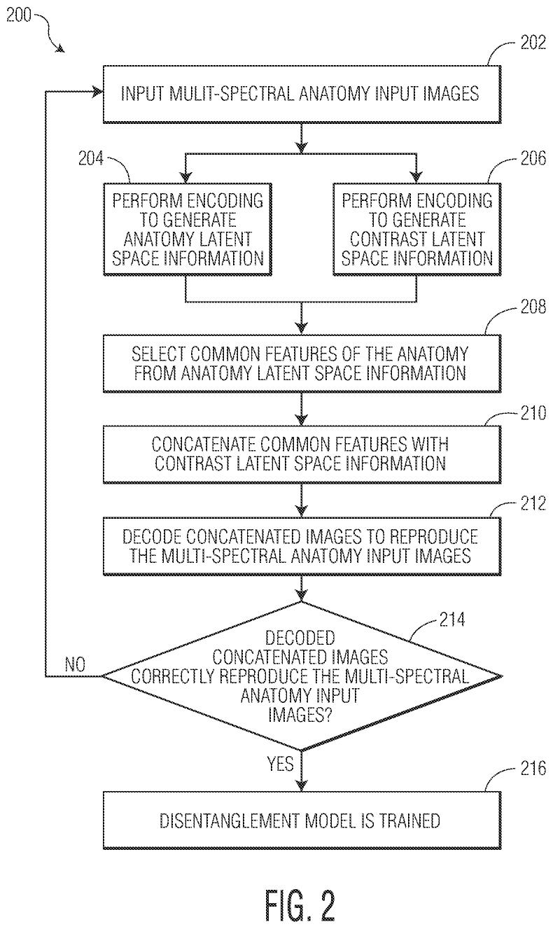

FIG. 2 shows a flowchart 200 of training the disentanglement model. In step 202, the multi-spectral images 202 are input to the disentanglement model. As mentioned above, multi-spectral images 202 may be images (e.g., CT images) of human anatomy captured at two or more energy levels (e.g., low energy level and high energy level). At steps 204 and 206, the disentanglement model performs encoding of the multi-spectral images 202. The encoding includes generating anatomy latent space information which represent compressed anatomy information in the multi-spectral images and generating contrast latent space information which represent compressed contrast information in the multi-spectral images. The encoded images are then input to a selector in step 208. The selector may be a random selector that select common anatomical features from the anatomy latent space information from any one of the energy levels. The selector within the disentanglement model plays a role in identifying and isolating the common anatomical features from the anatomy latent space information. These common features are the shared structural elements present across the multi-spectral images, regardless of the energy level at which each image was captured. The selector operates by analyzing the anatomy latent space information, which have been encoded to represent the core anatomical information, and then it identifies those features that remain invariant across different spectral frequencies. This process can be envisioned as a filter that sifts through the anatomy information, pinpointing the consistent elements that define the patient's anatomy.

Once the common anatomical features are selected, they serve as a stable base upon which the contrast information can be overlaid. The selector may employ a variety of techniques to accomplish this, such as statistical analysis to determine feature consistency. The output of the selector is a refined representation of the patient's anatomy, stripped of spectral variations but retaining the structural details. This distilled anatomy information is then concatenated with the contrast latent space information, which contain the energy-dependent attenuation information, to reconstruct the multi-spectral images or to synthesize new images that can be used for further analysis and diagnosis. At step 210, the common features output by the selector are then combined with the contrast latent space information. The images may be combined by separately concatenating (e.g., overlaying) the contrast latent space information with the common features output by the selector to produce two or more combined images which are input to respective decoders. In step 212, the decoders decoder the concatenated images in an attempt to reproduce the original multi-spectral images. In step 214, the method determines if the decoder correctly reproduced the original multi-spectral images or not. If the images are accurately reproduced, then the process moves on to step 216 where it is determined that the disentanglement model is accurately trained. If not, the process is repeated for a new set of multi-spectral images. The disentanglement model may be a neural network having weights that are adjusted until the decoder is able to correctly reproduce the original multi-spectral images from the latent space information.

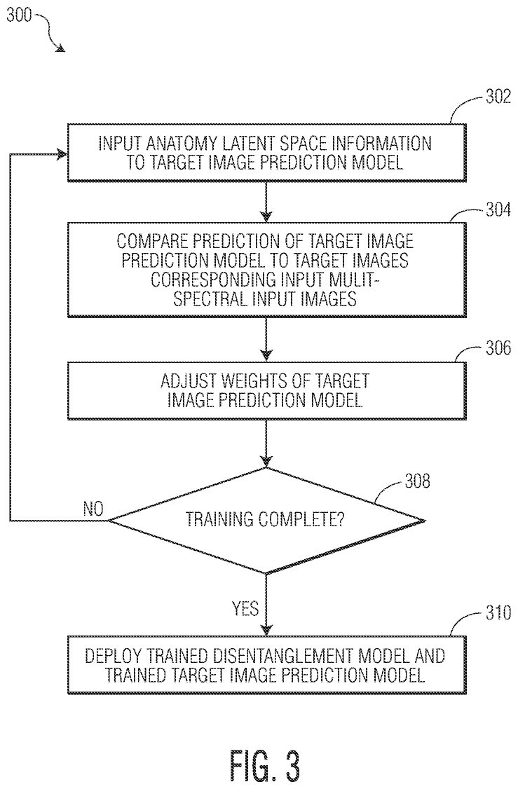

After the disentanglement model is accurately trained, the system then trains a prediction model for predicting desired target images. FIG. 3 shows a flowchart 300 of training the prediction model. In step 302, the system inputs the anatomy latent space information (compressed images) to the target image prediction model. Optionally, the anatomy latent space information 108A/108B (compressed images) may be pre-processed (e.g., averaged, etc.) prior to being input to the target image prediction model. In either case, in step 304, the system compares the prediction of the target image prediction model to known reference target images corresponding to the multi-spectral images. In other words, reference target images corresponding to the multi-spectral images are already known and may be compared to the prediction made by the target image prediction model. The target image prediction model may be a neural network having weights that are adjusted in step 306 based on the comparison between the labeled dataset. In step 308, the system determines if the training is complete or not. If the training is not complete (i.e., the predictions have not reached a desired level of accuracy), then the process is repeated for a new pair of labeled data. This process is repeated until training is complete, and in step 310 the system deploys the trained disentanglement model and target image prediction model. For case of deployment, the trained disentanglement model and target image prediction model may be combined as a single comprehensive model that performs disentanglement and then target image prediction based on the disentangled images.

As mentioned above, anatomy latent space can be learned in an unsupervised manner, which greatly reduces the amount of required training data. Furthermore, utilizing the common anatomy latent space as the input to the output decoder streamlines training of different types of prediction decoder algorithms without changing other parts of the networks. In other words, the anatomy disentanglement may be universal, but different prediction models can be developed for different types of target images. These prediction decoders may then be used in a “plug-and-play” fashion based on the targeted tasks. For example, special decoders for special types of medical predictions/diagnoses may be developed in a similar manner and then utilized as needed.

In other words, the training process for the spectral computed tomography system with disentangled representation is designed to be modular, allowing for the anatomy latent space to be learned in an unsupervised manner without the need to alter other components of the network. This modularity is achieved by first focusing on the disentanglement of anatomical and contrast features into separate latent spaces. The anatomy encoder is trained to capture the invariant anatomical structures across different energy levels, creating a universal representation of the patient's anatomy. This is done without supervision, meaning that the system does not require labeled data indicating the correct output for the anatomy latent space. The contrast encoder, on the other hand, learns the energy-dependent aspects of the images, which vary with the X-ray spectrum.

Once the anatomy latent space is established, it can be used as a consistent input for various prediction models, each designed for different target images such as virtual monoenergetic images, material maps, or segmentation maps. These prediction models are trained in a supervised manner, where they learn to map the anatomy latent space to the desired output. An advantage of this approach is that the anatomy latent space acts as a common foundation for all prediction models, which means that when a new prediction task arises or a new target image type is introduced, the existing anatomy encoder does not require retraining. Instead, a new prediction model is trained to work with the already established anatomy latent space. This “plug-and-play” capability streamlines the training process, as it allows for the addition of new prediction tasks with minimum adjustments to the overall network architecture, saving time and computational resources.

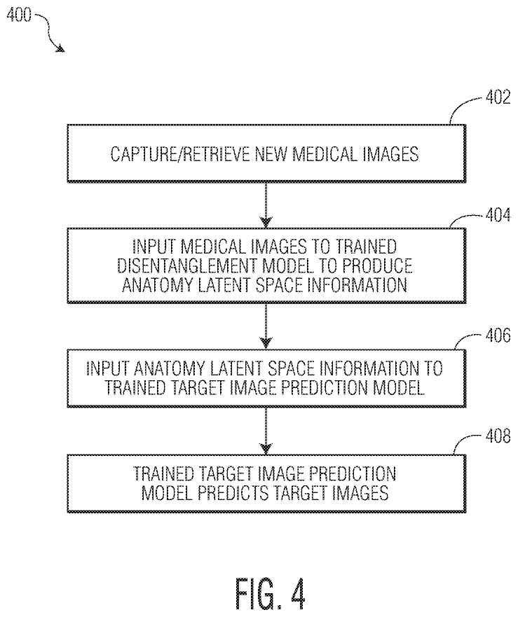

After the models are properly trained and deployed, the models may be used to perform target image prediction based on new sets of multi-spectral anatomy images. FIG. 4 shows a flowchart 400 of executing a trained models for spectral computed tomography with disentangled representation. For example, in step 402, the system may capture or retrieve new multispectral medical images. These images may be captured by a medical device (e.g., CT device) and input to the model or retrieved from storage of a user device or a database of medical images. In either case, the new images can be input to the trained disentanglement model to produce the anatomy latent space information in step 404. In step 406, the anatomy latent space information are then input to the trained target image prediction model. The trained image prediction model then can predict the target images in step 408.

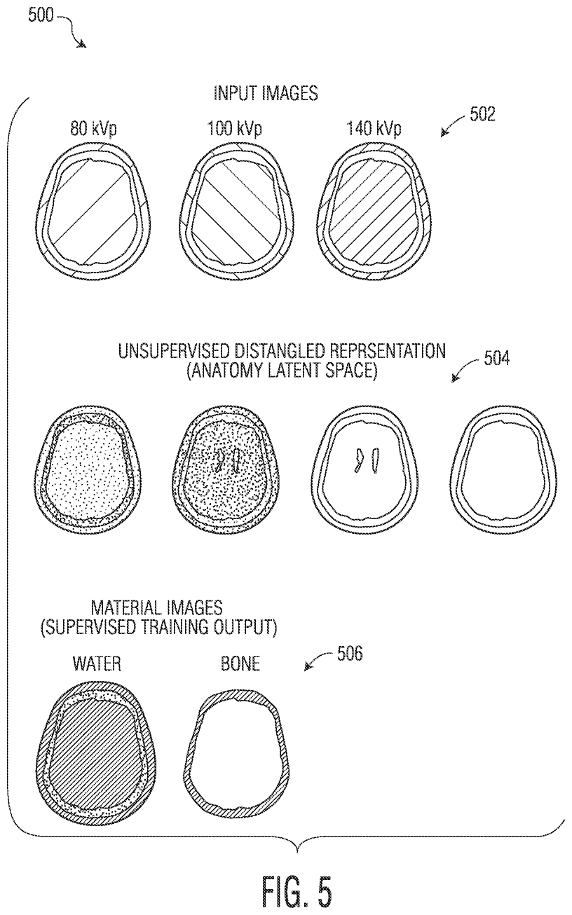

FIG. 5 illustrates example images 500 that demonstrate the process of spectral computed tomography with disentangled representation. Images 502 are the original input multi-spectral images obtained from a CT machine at varying energy levels. These images capture the anatomical details of the patient and the energy-dependent attenuation characteristics of the tissues. After the disentanglement process, images 504 represent the resultant anatomy latent space information, which may be compressed representations of the patient's anatomy, stripped of energy-specific contrast information. These images highlight the structural features of the anatomy that are consistent across different energy levels, such as the outlines of organs and bones. The anatomy latent space information are then used to predict target reference images 506, which are shown as material maps, but could be synthesized images that can include virtual monoenergetic images, or other clinically relevant visualizations. These target images are generated by decoding the anatomy latent space information to produce detailed and diagnostically useful representations of the patient's internal structures.

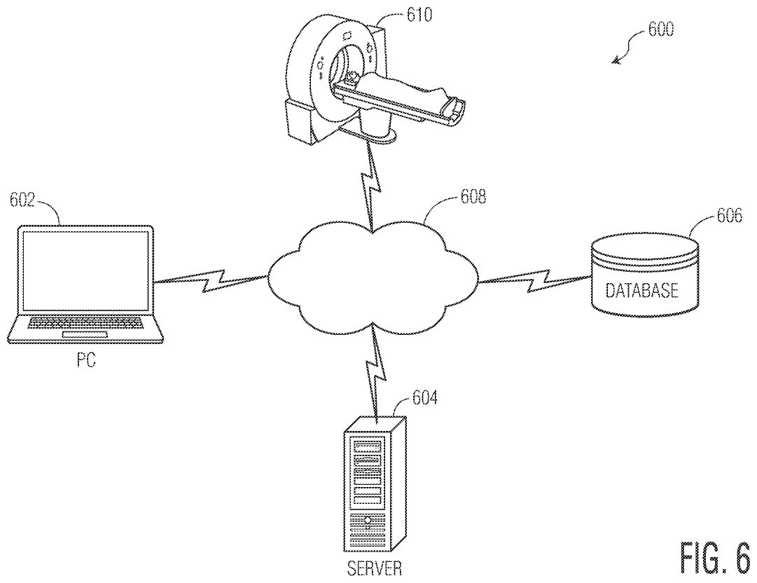

FIG. 6 shows a block diagram 600 of hardware devices for the training and execution of spectral computed tomography with disentangled representation. It should be understood that the components of the system 600 shown in FIG. 6 and described herein are merely examples and systems with additional, alternative, or fewer number of components should be considered within the scope of this disclosure.

As shown, system 600 comprises at least one end user device 602, server 604, database 606 and medical imaging device 610 interconnected through a network 608. In the illustrated example, server 604 supports operation (e.g., training, deployment and execution) of the spectral computed tomography with disentangled representation solution described herein. In the illustrated example, user device 602 is a PC, but may be any device (e.g., smartphone, tablet, etc.) providing access to the server 604 and database 606 via network 610. User device 602 has a user interface UI, which may be used to communicate with the server and database using the network 610 via a browser or via software applications. For example, user device 602 may allow the user to access trained models executing on server 604, while server 604 may be used to train and deploy the models. The network 608 may be the Internet and or other public or private networks or combinations thereof. The network 608 therefore should be understood to include any type of circuit switching network, packet switching network, or a combination thereof. Non-limiting examples of the network 608 may include a local area network (LAN), metropolitan area network (MAN), wide area network (WAN), and the like.

In an example, end user device 602 may communicate with server 604 via a software application to access the models disclosed herein. The software application may initiate server 604 to execute the trained model. For example, server 604 may receive medical images from one or more of user device 602, database 606 and medical device 610. Server 604 may then train the disengagement model and prediction models based on these images. Server 604 may then deploy the trained model and allow the user to access and execute the trained model via user device 602 for new medical images.

Devices 602, 604, 606 and 610 are each depicted as single devices for case of illustration, but those of ordinary skill in the art will appreciate that devices 602, 604, 606 and 610 may be embodied in different forms for different implementations. For example, any or each of the servers may include a plurality of servers including a plurality of databases, etc. Alternatively, the operations performed by any of the servers may be performed on fewer (e.g., one or two) servers. In another example, a plurality of user devices (not shown) may communicate with the servers. Furthermore, a single user may have multiple user devices (not shown), and/or there may be multiple users (not shown) each having their own respective user devices (not shown). Regardless, the hardware configuration shown in FIG. 6 may be a system that supports the functionality of the model training and execution disclosed herein.

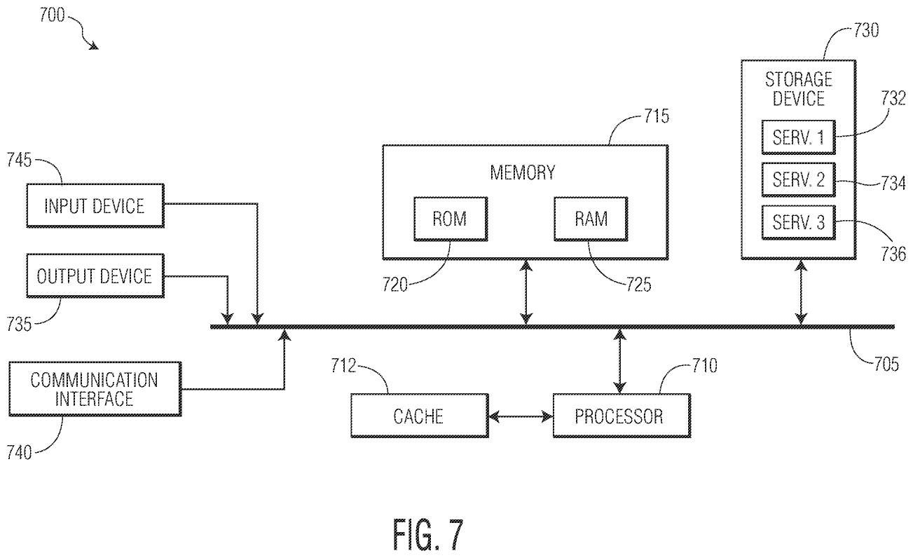

FIG. 7 shows a block diagram of components of the training and execution of hardware devices for spectral computed tomography with disentangled representation. System 700 may be representative of at least a portion of each of PC 602, server 604, database 606 and medical imaging system 610. One or more components of system 700 may be in electrical communication with each other using a bus 705. System 700 may include a processing unit (CPU or processor) 710 and a system bus 705 that couples various system components including the system memory 715, such as read only memory (ROM) 720 and random-access memory (RAM) 725, to processor 710. System 700 may include a cache of high-speed memory connected directly with, in close proximity to, or integrated as part of processor 710. System 700 may copy data from memory 715 and/or storage device 730 to cache 712 for quick access by processor 710. In this way, cache 712 may provide a performance boost that avoids processor 710 delays while waiting for data. These and other modules may control or be configured to control processor 710 to perform various actions. Other system memory 715 may be available for use as well. Memory 715 may include multiple different types of memory with different performance characteristics. Processor 710 may include any general-purpose processor and a hardware module or software module, such as service 1 732, service 2 734, and service 3 736 stored in storage device 730, configured to control processor 710 as well as a special-purpose processor where software instructions are incorporated into the actual processor design. Processor 710 may essentially be a completely self-contained computing system, containing multiple cores or processors, a bus, memory controller, cache, etc. A multi-core processor may be symmetric or asymmetric.

To enable user interaction with the computing system 700, an input device 745 may represent any number of input mechanisms, such as a microphone for speech, a touch-sensitive screen for gesture or graphical input, keyboard, mouse, motion input, speech and so forth. An output device 735 may also be one or more of a number of output mechanisms known to those of skill in the art. In some instances, multimodal systems may enable a user to provide multiple types of input to communicate with computing system 700. Communications interface 740 may generally govern and manage the user input and system output. There is no restriction on operating on any particular hardware arrangement and therefore the basic features here may easily be substituted for improved hardware or firmware arrangements as they are developed.

Storage device 730 may be a non-volatile memory and may be a hard disk or other types of non-transitory computer readable media which may store data that are accessible by a computer, such as magnetic cassettes, flash memory cards, solid state memory devices, digital versatile disks, cartridges, random access memories (RAMs) 725, read only memory (ROM) 720, and hybrids thereof.

Storage device 730 may include services 732, 734, and 736 for controlling the processor 710. Other hardware or software modules are contemplated. Storage device 730 may be connected to system bus 705. In one aspect, a hardware module that performs a particular function may include the software component stored in a computer-readable medium in connection with the necessary hardware components, such as processor 710, bus 705, output device 735, and so forth, to carry out the function.

While the foregoing is directed to example embodiments described herein, other and further example embodiments may be devised without departing from the basic scope thereof. For example, aspects of the present disclosure may be implemented in hardware or software or a combination of hardware and software. One example embodiment described herein may be implemented as a program product for use with a computer system. The program(s) of the program product defines functions of the example embodiments (including the methods described herein) and may be contained on a variety of computer-readable storage media. Illustrative computer-readable storage media include, but are not limited to: (i) non-writable storage media (e.g., read-only memory (ROM) devices within a computer, such as CD-ROM disks readably by a CD-ROM drive, flash memory, ROM chips, or any type of solid-state non-volatile memory) on which information is permanently stored; and (ii) writable storage media (e.g., floppy disks within a diskette drive or hard-disk drive or any type of solid-state random-access memory) on which alterable information is stored. Such computer-readable storage media, when carrying computer-readable instructions that direct the functions of the disclosed example embodiments, are example embodiments of the present disclosure.

It will be appreciated by those skilled in the art that the preceding examples are exemplary and not limiting. It is intended that all permutations, enhancements, equivalents, and improvements thereto are apparent to those skilled in the art upon a reading of the specification and a study of the drawings are included within the true spirit and scope of the present disclosure. It is therefore intended that the following appended claims include all such modifications, permutations, and equivalents as fall within the true spirit and scope of these teachings.

What is claimed:

1. A system for generating target images, comprising:
a database of multi-spectral images; and
a processor configured to:
train a latent space encoder to generate latent space information by:
encoding the multi-spectral images to generate anatomy latent space information and contrast latent space information, the anatomy latent space information representing compressed anatomy information in the multi-spectral images, the contrast latent space information representing compressed contrast information in the multi-spectral images,
combining selected features of the anatomy latent space information and the contrast latent space information to reproduce the multi-spectral images,
comparing the reproduced multi-spectral images to the multi-spectral images, and
adjusting the encoding of the multi-spectral images and repeating the training until the reproduced multi-spectral images match the multi-spectral images, and
after training the latent space encoder, train an output decoder to generate target images by:
inputting the anatomy latent space information to a prediction model,
predicting target images by the prediction model based on the anatomy latent space information,
comparing the predicted target images to reference target images related to the multi-spectral images, and
adjusting weights of the prediction model based on the comparison and repeating the training until the images predicted by the prediction model match the reference target images related to the multi-spectral images.

2. The system of claim 1, wherein the processor is configured to generate the latent space information by applying a convolutional neural network to the multi-spectral images to extract and separate anatomy and contrast information from the multi-spectral images.

3. The system of claim 1, wherein the processor is configured to select anatomy features as common anatomy features between the anatomy latent space information.

4. The system of claim 1, wherein the processor is configured to combine the anatomy latent space information and the contrast latent space information by concatenating the anatomy latent space information and the contrast latent space information.

5. The system of claim 1, wherein the processor is configured to compare the reproduced multi-spectral images to the multi-spectral images by computing a loss function of the reproduced multi-spectral images as compared to the multi-spectral images and repeating the training until the loss function is less than a loss function threshold.

6. The system of claim 1, wherein the multi-spectral images are images of anatomy captured by medical imaging devices from a common frame of reference relative to the anatomy and operating at different spectral frequencies.

7. The system of claim 1, wherein the processor is configured to average the anatomy latent space information prior to inputting the anatomy latent space information to the prediction model.

8. The system of claim 1, wherein the prediction model is a neural network that predicts the target images and in supervised manner compares the predicted target images to reference target images related to the multi-spectral images to determine corrective measures to adjust the weights of the prediction model which are neural network weights.

9. The system of claim 1, wherein the target images are medical images related to states of human anatomy and comprise one or more of virtual monoenergetic images, virtual non-contrast images, material maps and downstream segmentations.

10. The system of claim 1, wherein the processor is configured to utilize the trained latent space encoder to generate new latent space information for new multi-spectral images and input the new latent space information to the trained output decoder to generate new target images.

11. A method for generating target images, comprising:
training, by a processor, a latent space encoder to generate latent space information by:
encoding multi-spectral images to generate anatomy latent space information and contrast latent space information, the anatomy latent space information representing compressed anatomy information in the multi-spectral images, the contrast latent space information representing compressed contrast information in the multi-spectral images,
combining selected features of the anatomy latent space information and the contrast latent space information to reproduce the multi-spectral images,
comparing the reproduced multi-spectral images to the multi-spectral images, and
adjusting the encoding of the multi-spectral images and repeating the training until the reproduced multi-spectral images match the multi-spectral images, and
after training the latent space encoder, training, by the processor, an output decoder to generate target images by:
inputting the anatomy latent space information to a prediction model,
predicting target images by the prediction model based on the anatomy latent space information,
comparing the predicted target images to reference target images related to the multi-spectral images, and
adjusting weights of the prediction model based on the comparison and repeating the training until the images predicted by the prediction model match the reference target images related to the multi-spectral images.

12. The method of claim 11, generating, by the processor, the latent space information by performing convolutions on the multi-spectral images to extract and separate anatomy and contrast information from the multi-spectral images.

13. The method of claim 11, selecting, by the processor, anatomy features as common anatomy features between the anatomy latent space information.

14. The method of claim 11, combining, by the processor, the anatomy latent space information and the contrast latent space information by concatenating the anatomy latent space information and the contrast latent space information.

15. The method of claim 11, comparing, by the processor, the reproduced multi-spectral images to the multi-spectral images by computing a loss function of the reproduced multi-spectral images as compared to the multi-spectral images and repeating the training until the loss function is less than a loss function threshold.

16. The method of claim 11, wherein the multi-spectral images are images of anatomy captured by medical imaging devices from a common frame of reference relative to the anatomy and operating at different spectral frequencies.

17. The method of claim 11, averaging, by the processor, the anatomy latent space information prior to inputting the anatomy latent space information to the prediction model.

18. The method of claim 11, wherein the prediction model is a neural network that predicts the target images and in supervised manner compares the predicted target images to reference target images related to the multi-spectral images to determine corrective measures to adjust the weights of the prediction model which are neural network weights.

19. The method of claim 11, wherein the target images are medical images related to states of human anatomy and comprise one or more of virtual monoenergetic images, virtual non-contrast images, material maps and downstream segmentations.

20. The method of claim 11, utilizing, by the processor, the trained latent space encoder to generate new latent space information for new multi-spectral images and input the new latent space information to the trained output decoder to generate new target images.
